(19) United States
(12) Patent Application Publication
(10) Pub. No.: US 2017/0056237 A1
(43) Pub. Date: Mar. 2, 2017

(54) SYSTEM AND METHOD FOR FALLOPIAN TUBE OCCLUSION

(71) Applicant: CONTRAMED, LLC, San Jose, CA (US)

(72) Inventor: Michael Tal, Savyon (IL)

(21) Appl. No.: 14/841,971

(22) Filed: Sep. 1, 2015

(51) Int. Cl. A61F 6/20; A61M 25/00; A61B 17/3211; A61M 25/01; A61B 17/42; A61F 6/06; A61M 5/14

(52) U.S. Cl. CPC A61F 6/20; A61F 6/06; A61M 25/0082; A61M 5/14; A61M 25/0097; A61M 25/0108; A61B 17/42; A61B 17/3211; A61B 2017/4233

(57) ABSTRACT

A system for treating a fallopian tube to promote contraception includes a catheter device, a tissue disrupting head coupled with the catheter device, and a fluid delivery port for delivering a sclerosant to the mucosal lining of the fallopian tube before, during or after disruption of the mucosal lining by the tissue disrupting head. The catheter device may include a handle and a catheter shaft having a proximal end coupled with the handle and a distal end sized and configured to be advanced through a cervix and into the fallopian tube. The tissue disrupting head may include at least one tissue disrupting member configured to operable to mechanically disrupt a mucosal lining of the fallopian tube by contacting the tissue disrupting member with the mucosal lining and moving the tissue disrupting head. The fluid delivery port may be located in the catheter shaft or the tissue disrupting head.

### TECHNICAL FIELD

The present application is related to medical devices, systems and methods. More specifically, the application is related to a system and method for occluding fallopian tubes to promote contraception.

### BACKGROUND

Birth control, also known as contraception or fertility control, encompasses methods and devices for the prevention of pregnancy. Birth control has been practiced since ancient times, but effective and safe approaches only became available in the 20th century.

Birth control can be carried out using barrier approaches (e.g., condoms, diaphragm), hormonal approaches (e.g., contraceptive pills or emergency contraceptive pills), intrauterine devices (IUDs) or sterilization (e.g., vasectomy in males and tubal ligation in females). Tubal ligation is a surgical procedure, in which the fallopian tubes are cut, tied or blocked. Tubal ligation is considered permanent and effective in preventing pregnancies, but it involves major surgery and hospitalization and carries a risk of infection and other complications.

Blocking the fallopian tubes (also referred to as “fallopian tube occlusion”) is typically effected via tubal implants, such as the Essure® permanent contraceptive device. The Essure® device consists of a small metal spring that is placed within the fallopian tube. Over time, scar tissue grows around the device to permanently block the fallopian tube. Implants for blocking the fallopian tube can be inserted by catheter in a doctor's office, without anesthesia or incisions, with most patients returning to normal activities in one or two days. Prolonged use of tubal implants, however, can lead to serious complications. For example, the Essure® device, which has been on the market since 2002, has been recently associated with serious complications, including severe back and pelvic pain, heavy prolonged menstrual periods, and device dislodgement, resulting in coils piercing through the fallopian tubes and sometimes even migrating to other organs.

Therefore, a need exists for improved methods and systems for obstructing fallopian tubes to help provide contraception. Ideally, such methods and systems would be minimally invasive and would avoid at least some of the shortcomings and potential complications of tubal ligation procedures and currently available fallopian tube occluding implants. At least some of these objectives will be addressed by the embodiments described herein.

### BRIEF SUMMARY

Generally, embodiments of a system and method described herein are configured to mechanically disrupt the mucosal lining of a fallopian tube and deliver a sclerosant to the mucosal lining to induce the formation of a tissue plug within an intramural region of the fallopian tubes. In some embodiments, a system for treating fallopian tubes may include: (a) a catheter sized and configured for transcervical delivery; (b) a tissue disrupting head mounted in or on the catheter and operable to mechanically disrupt a mucosal lining of a fallopian tube; and (c) a fluid delivery port on the catheter or the tissue disrupting head, for delivering a sclerosant to the mucosal lining of the fallopian tube before, during and/or after operation of the tissue disrupting head.

In one aspect, a system for treating a fallopian tube to promote contraception includes a catheter device, a tissue disrupting head and at least one fluid port. The catheter device includes a handle and a catheter shaft having a proximal end coupled with the handle and a distal end sized and configured to be advanced through a cervix and into the fallopian tube. The tissue disrupting head is coupled with the catheter and includes at least one tissue disrupting member configured to mechanically disrupt a mucosal lining of the fallopian tube by contacting the tissue disrupting member with the mucosal lining and moving the tissue disrupting head. The fluid delivery port (or ports) is located in the catheter shaft and/or the tissue disrupting head, and is used for delivering a sclerosant to the mucosal lining of the fallopian tube before, during and/or after disruption of the mucosal lining by the tissue disrupting head.

In some embodiments, the tissue disrupting head is slidably disposed in a lumen of the catheter shaft, so that it slides in and out of a distal opening of the catheter shaft. In some embodiments, the tissue disrupting member(s) are configured to disrupt the mucosal lining when the tissue disrupting head is moved back and forth relative to the fallopian tube. In some embodiments, the handle includes a sliding handle portion coupled with the tissue disrupting head, so that moving the sliding handle portion back and forth relative to the handle causes the tissue disrupting head to move in and out of the distal opening of the catheter shaft. Optionally, the sliding handle portion may be configured to rotate relative to the handle to cause the tissue disrupting head to rotate relative to the catheter shaft. In such embodiments, tissue disrupting member(s) may be configured to disrupt the mucosal lining when the tissue disrupting head is rotated. In some embodiments, the sliding handle portion may be coupled with the tissue disrupting head via a tube slidably disposed within a lumen of the catheter shaft. In other embodiments, the tissue disrupting head may be a distal portion of a tube slidably disposed within a lumen of the catheter shaft and attached at a proximal end to the sliding handle portion.

Optionally, the system may also include a stop on an inner surface of the catheter shaft for stopping distal movement of the tissue disrupting head beyond a predetermined distance out of the distal opening of the catheter shaft. The system may also optionally include a guidewire lumen extending through at least a portion of the catheter shaft. Such an embodiment may also include a tube disposed within a lumen of the catheter shaft and connecting the tissue disrupting head to the handle, and the guidewire lumen may be an inner lumen of the tube that extends from a proximal opening in the handle to a distal opening in the tissue disrupting head.

In some embodiments, the fluid delivery port may be a distal opening in the tissue disrupting head that is in fluid communication with a lumen of a tube extending proximally from the tissue disrupting head. Some embodiments may include multiple fluid ports in a wall of the catheter body, while other embodiments may include multiple fluid ports in the tissue disrupting head.

The system may also optionally include a quantity of the sclerosant for delivery through the fluid delivery port(s). The tissue disrupting head, in some embodiments, may include a tissue reservoir for collecting at least some of the mucosal lining that is disrupted. In some embodiments, the system includes at least one imaging marker on the catheter shaft for facilitating assessment of a location of the catheter shaft relative to the fallopian tube. Some embodiments may also include a tube slidably disposed within a lumen of the catheter shaft, and the tube may include at least one additional imaging marker. In some embodiments, the catheter device may further include a positioning member on an outer surface of the catheter shaft near its distal end. The positioning member is configured to prevent advancement of the distal end of the catheter shaft past a desired location in the fallopian tube. The tissue disrupting member(s) may include any of a number of suitable mechanisms for tissue disruption, such as but not limited to wires, coils, tines with attached balls and/or blades.

In another aspect, a method of occluding a fallopian tube may involve: advancing a distal end of a catheter into the fallopian tube; positioning a tissue disrupting head coupled with the catheter in an intramural region of the fallopian tube; moving the tissue disrupting head within the intramural region to mechanically disrupt a mucosal lining of the fallopian tube; and delivering a sclerosant to the mucosal lining of the fallopian tube. In some embodiments, advancing the distal end of the catheter involves advancing it through the cervix to access the fallopian tube. In some embodiments, positioning the tissue disrupting head comprises advancing the tissue disrupting head out of the distal end of the catheter. In some embodiments, advancing the tissue disrupting head out of the distal end of the catheter allows one or more tissue disrupting members on the tissue disrupting head to expand from a constrained configuration to an expanded configuration for disrupting the tissue.

Moving the tissue disrupting head may involve moving it in one or more directions, such as proximally relative to the fallopian tube, distally relative to the fallopian tube, back and forth, or rotating the tissue disrupting head. In various embodiments, delivering the sclerosant may be performed before, during and/or after moving the tissue disrupting head to disrupt the mucosal lining.

These and other aspects and embodiments are described in greater detail below, in relation to the attached drawing figures.

### BRIEF DESCRIPTION OF THE DRAWINGS

FIG. 1 is a partial cross-section, front anatomical illustration of a uterus and fallopian tubes;

FIGS. 2A and 2B are side and cross-sectional views, respectively, of a fallopian tube occlusion system, according to one embodiment;

FIG. 2C is a magnified version of a distal portion of the system, which is circled in FIG. 2B;

FIG. 3A is a perspective view of a portion of a fallopian tube occlusion system, including a tissue disrupting head, according to one embodiment;

FIG. 3B is a perspective view of a portion of a fallopian tube occlusion system, including a tissue disrupting head, according to an alternative embodiment;

FIG. 3C is a perspective view of a portion of a fallopian tube occlusion system, including a tissue disrupting head, according to one embodiment; and

FIGS. 4A-4G are side, cross-sectional views of a uterus and fallopian tubes, illustrating a method for delivering a tissue disrupting head to the intramural region of the fallopian tubes, according to one embodiment.

### DETAILED DESCRIPTION

Embodiments of a fallopian tube occlusion system and method are described herein. Specifically, the fallopian tube occlusion system and method may be used to occlude the intramural region of a fallopian tube, using a minimally invasive approach that avoids the use of potentially harmful implants. Although a number of embodiments and details are described, the scope of the invention should not be interpreted as being limited to the embodiments and details set forth in the following description or illustrated in the drawings.

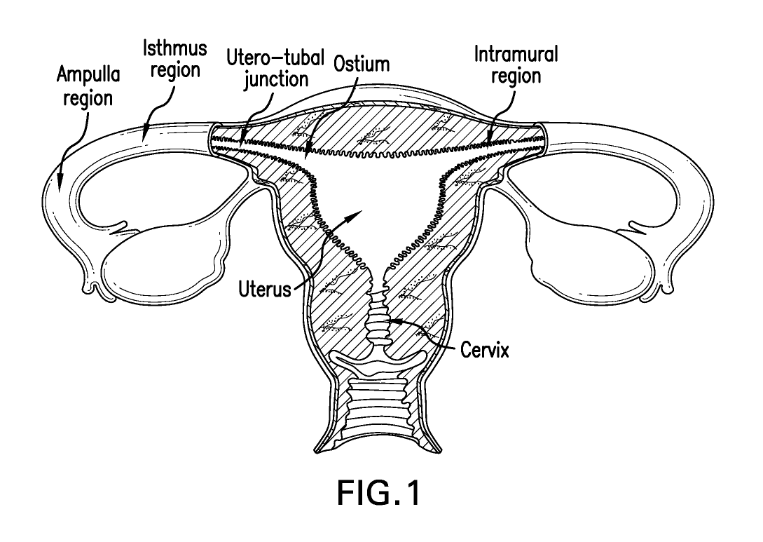

FIG. 1 illustrates the general anatomy of the uterus and fallopian tubes. A fallopian tube is typically described as having three regions: the ampulla region, which represents the major portion of the lateral tube; the isthmus region, which is the narrower portion of the tube that links to the uterus; and the intramural region (also known as the interstitial region), which transverses the uterine musculature. The tubal ostium is the point where the tubal canal meets the peritoneal cavity. The uterine opening of the fallopian tube is the entrance into the uterine cavity at the utero-tubal junction.

Approaches for blocking the fallopian tubes via implants target the intramural region of the fallopian tube, since this region of the tubes is surrounded by the uterine musculature and is thus the most suited for anchoring implants. Displacement of implants within the fallopian tube, however, has led to piercing of fallopian tube walls and in extreme cases migration of implants to other organs.

Rather than implanting an implant in a fallopian tube, the system and method described herein occlude a fallopian tube by disrupting fallopian tube tissue and delivering a sclerosant to the disrupted tissue to form a natural tissue plug within the intramural region of the fallopian tube at or near the ostium. The natural tissue plug will occlude the fallopian tube and thus help promote contraception (e.g., help prevent unwanted pregnancy). Generally, the system includes a catheter configured for transcervical delivery into the uterus and fallopian tubes, a tissue disrupting head mounted in or on the catheter, and a fluid delivery port on the catheter or tissue disrupting head for delivering a sclerosant to the mucosal lining of the fallopian tube prior to, during and/or after tissue disruption. The tissue disrupting head is designed to mechanically disrupt the mucosal lining of a fallopian tube, preferably at the intramural region.

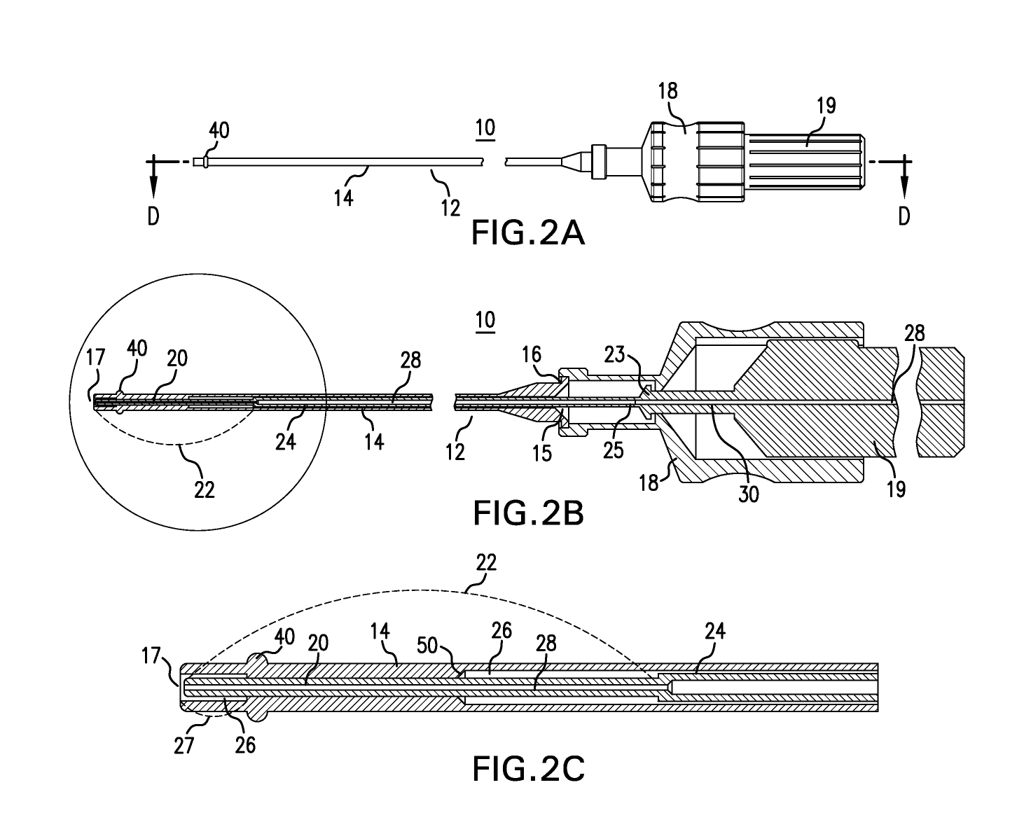

Referring now to FIGS. 2A-2C, one embodiment of a fallopian tube occlusion system 10 will be described in greater detail. In this embodiment, system 10 includes a delivery catheter device 12 and a tissue disrupting head 20 disposed inside catheter device 12. Catheter device 12 generally includes a shaft 14, a handle 18, and a tube 24 disposed within shaft 14. Shaft 14 includes: a proximal end 16, with a proximal opening 15; a distal opening 17; a lumen 26 (FIG. 2C) formed by its inner wall; and a positioning member 40 on its outer wall near the distal opening 17. Tube 24 may be slidably disposed within lumen 26, such that it can advance and retract tissue disrupting head 20 out of distal end 17. Tube 24 also forms an inner, guidewire lumen 28. Tissue disrupting head 20 forms a distal portion 22 of tube 24, although in alternative embodiments it may be a separate piece attached to the distal end of tube 24. Tissue disrupting head 20 is disposed within lumen 26 of shaft 14 and is slidable in and out of distal opening 17 via push/pull of a proximal end 25 of tube 24. In alternative embodiments, tissue disrupting head 20 may be mounted on or around shaft 14. A distal lumen portion 27 of lumen 26, immediately proximal to opening 17, may have a larger inner diameter than the rest of lumen 26, to accommodate the larger diameter tissue disrupting head 20 when sequestered therein.

In various embodiments, shaft 14 of catheter device 12 may have a length of about 200-500 mm and an outer diameter (OD) of about 2-5 mm. Proximal end 16 of shaft 14 is attached to handle 18 for operating catheter device 12 and tissue disrupting head 20 disposed in shaft 14. Shaft 14 may be fabricated from a polymer, such as Pebax, nylon, polyimide, PTFE and the like, an alloy, such as stainless steel, Nitinol, chromium-cobalt and the like, a combination of both (e.g., an alloy-braided polymer shaft) using molding, extrusion or machining approaches, or any other suitable material. Shaft 14 is preferably flexible (elastically bendable), such that it can be guided transvaginally through the cervix and intrauterine cavity and into a fallopian tube. Such guiding can be effected using a guidewire in some embodiments, in which case catheter shaft 14 will include guidewire lumen 28, running a length of shaft 14 from a proximal opening 15 to distal opening 17. Guidewire lumen 28 may have a diameter of about 0.4-4 mm, thereby enabling use of a guidewire having a diameter of about 0.014″-0.035″. In some embodiments, guidewire lumen 28 may also be used to deliver tissue disruption head 20 to the target tissue. In alternative embodiments, lumen 26 of shaft 14 (in which tube 24 is positioned) may serve as a guidewire lumen.

Optionally, catheter shaft 14 may be steerable via wires disposed within dedicated lumens running a length of shaft 14. Such wires may be actuated from handle 18. Examples of such steerable catheter shafts, any of which may be incorporated into system 10, are described in U.S. Pat. Nos. 5,507,725 and 8,700,120.

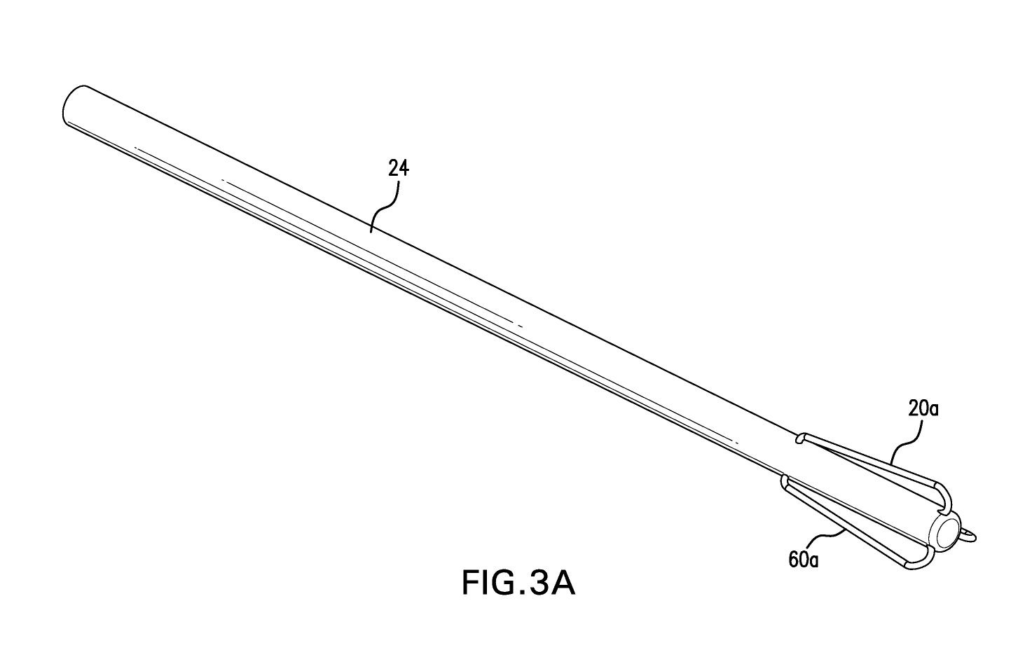

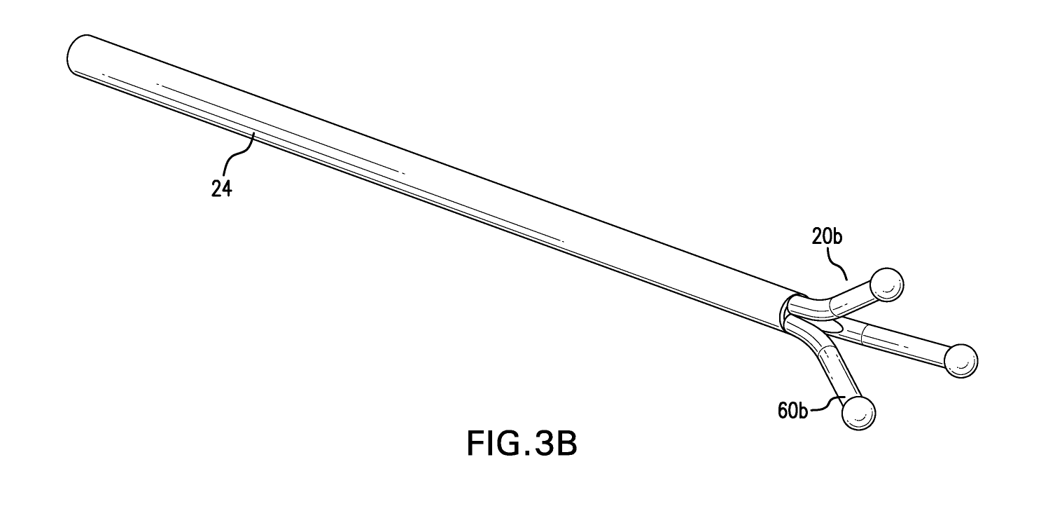

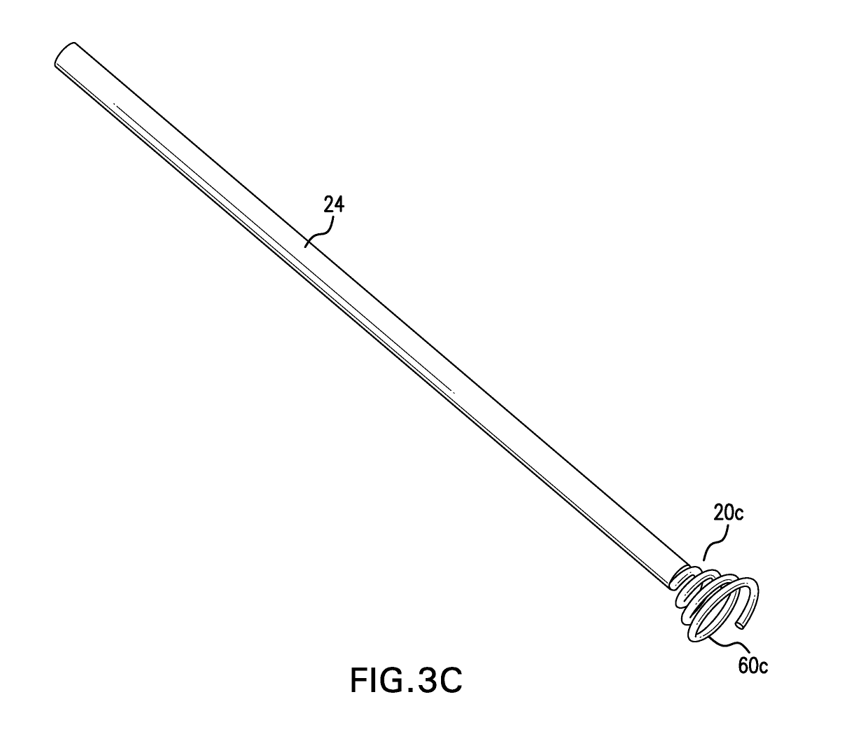

Tube 24, which extends through the inside of shaft 14, is preferably about 200-500 mm in length and has an OD of about 0.5-4 mm. Tissue disrupting head 20 may have a length of about 1-20 mm and includes one or more tissue engaging elements (shown and described in subsequent figures), which extend radially outward a distance of about 2-10 mm. These elements can be fabricated from a polymer, such as nylon, PEEK, polycarbonate, ABS and the like, or an alloy, such as stainless steel, Nitinol, chromium-cobalt and the like. Tissue disruption head 20 is preferably configured for ripping, rather than cutting, fallopian wall tissue (e.g., the mucosal layer). In alternative embodiments, however, tissue disrupting head 20 may include cutting blades. Tissue disrupting head 20 may disrupt tissue when pulled, pushed and/or spun, depending on the configuration of the tissue engaging elements. These features are discussed more fully below, in relation to FIGS. 3A-3C.

In the embodiment shown in FIGS. 2A-2C, tissue disrupting head 20 is rotatable to disrupt fallopian tube tissue, and as such, proximal end 25 of tube 24 is attached to a motorized drive mechanism 30 disposed within handle 18. Tissue disrupting head 20 may be rotated at about 500-5000 RPM, for example, via a direct or geared drive shaft.

Since tube 24 also functions in moving tissue disrupting head 20 in and out of opening 17, attachment of tube 24 to drive mechanism 30 is effected in a manner that enables both rotation and translation of tissue disrupting head 20 within shaft 14. Such an attachment may be realized by connecting a proximal non-circular portion of tube 24 (e.g., square or hexagonal) within or around a complementary coupler in drive mechanism 30. Such attachment allows for transfer of torque from drive mechanism 30 to tube 24, while also allowing tube 24 to slide back and forth within lumen 26 of shaft 14. Alternative embodiments may include different connecting mechanisms to connect tube 24 to drive mechanism 30, such as couplings that engage only when tube 24 is pushed forward to advance tissue disrupting head 20 out of shaft 14.

Handle 18 may include an inner, slidable portion 19, which may include or be attached to drive mechanism 30 and a distal engagement feature 23. To advance tissue disrupting head 20 out of distal opening 17 and optionally engage drive mechanism 30 with tube 24, a user may hold handle 18 while pushing slidable portion 19 forward (in the distal direction) until distal engagement feature 23 engages proximal engagement feature 25 at proximal opening 15 of shaft 14. This can serve as a drive engagement mechanism and also as an alternative or supplemental element for tissue disrupting head 20. In some embodiments, guidewire lumen 28 may extend through slidable portion 19, drive mechanism 30 and distal engagement feature 23, thus extending all the way through to the proximal end of handle 18.

Handle 18 may also include a power source (e.g. battery) for powering drive mechanism 30 and one or more controllers for actuating drive mechanism 30, setting the speed of rotation of tube 24 (and tissue disrupting head 20), and moving tissue disrupting head 20 in and out of shaft 14.

To prevent improper positioning of system 10 during a fallopian tube occlusion procedure, system 10 also includes positioning element 40, disposed on the outer surface of shaft 14, approximately 5-30 mm from distal opening 17. Positioning element 40 may be a cuff (as shown), formed as a lateral extension of the outer wall of shaft 14 and having an OD of about 4-15 mm. In alternative embodiment, positioning element 40 may instead be fixedly attached to the outer wall of shaft 14 via adhesive or mechanical fasteners, or it may be an inflatable toroidal balloon. In yet other embodiments, positioning element 40 may be movable along a length of shaft 14 and locked thereto (e.g., via pin) at one of several positions, based on the preferences of the user or the anatomy of the patient. Since the diameter of the fallopian tube opening/ostium ranges between about 1-3, positioning element 40 helps prevent advancement of shaft 14 into the fallopian tube beyond positioning element 40.

System 10 may also include an inner stop 50 (FIG. 2C), for limiting the distance tissue disrupting head 20 can be advanced out of shaft 14. Stop 50 can be a narrowing in lumen 26 which serves to prevent tube 24 from further distal advancement. In an alternative embodiment, stop 50 may be replaced by a lock mechanism in handle 18.

Fallopian tube occlusion system 10 is generally configured for disrupting tissue in a fallopian tube, to stimulate a tissue response that will cause formation of a tissue plug that blocks the tube, thus promoting contraception. In some embodiments, system 10 is specifically configured to facilitate fallopian tube tissue treatment and occlusion at a specific region of the fallopian tube (e.g., intramural region). Alternative embodiments may be directed toward treatment and occlusion of a different portion of a fallopian tube. To directly target the intramural region of the fallopian tube, system 10 includes positioning element 40 and stop 50, which help ensure that catheter shaft 14 and the tissue disrupting head 20 are accurately positioned prior to tissue disruption and sclerosant delivery.

Although positioning element 40 and stop 50 may facilitate use of system 10 without the need for imaging devices, system 10 may also include one or more imaging markers. For example, shaft 14 may include two or more circumferential (band-like) imaging markers (e.g., radiopaque (gold or platinum) or ultrasound markers), spaced apart about 10-40 mm from the distal tip. Tube 24 may include a single marker, for example, positioned in between the markers on shaft 14. Such markers help an operator to image the working tip of catheter device 12 and ascertain the exact position of shaft 14 and tissue disrupting head 20.

System 10 also includes at least one fluid delivery port for delivering a sclerosant to the site of fallopian tube tissue disruption. In one embodiment, for example, distal opening 17 of shaft 14 may act as the fluid delivery port, in which case sclerosant may be delivered via lumen 16 (around tube 24). Alternatively, the distal opening of tube 24 may act as the fluid delivery port, in which case the sclerosant may be delivered through guidewire lumen 28. In yet other alternative embodiments, one or more fluid delivery ports may be formed as separate port(s) in a distal portion of shaft 14 and/or a distal portion of tissue disrupting head 20. In such embodiments, the separate fluid delivery port(s) may either be in fluid communication with one of the two lumens 26, 28, or system 10 may include one or more additional fluid conduits leading to the port(s), with such conduits running through shaft 14 and/or tube 24, for example. Any suitable sclerosant may be used with/delivered by system 10, according to various embodiments. Sclerosant may be provided in fluid or gel form and may include a mucoadhesive and/or other additive(s) (e.g., contrast material). Examples of suitable sclerosant include, but are not limited to, Polydocanol, Sutradecol and alcohol. The sclerosant may be delivered to system 10 by any suitable means, such as via a syringe fitted to a port (not shown) on shaft 14 or handle 18. In any case, the proximal end of the sclerosant conduit can be connected to a conduit within handle 18 which in turn can be connected to a sclerosant source (e.g. syringe) via, for example, a Luer lock.

Referring now to FIGS. 3A-3C, three alternative embodiments of tissue disrupting head 20a, 20b, 20c include three different types of tissue disrupting members 60a, 60b, 60c. In all three embodiments, tissue disrupting head 20a, 20b, 20c is simply a distal portion of tube 24, onto which tissue disrupting members 60a, 60b, 60c are mounted. In alternative embodiments (not illustrated), tissue disrupting head 20 may instead be a separate piece that is attached to a distal end of tube 24.

FIG. 3A illustrates a tissue disrupting head 20a that includes three tissue disrupting members 60a shaped as trapezoidal loops. According to various embodiments, each tissue disrupting member 60a may be a wire, made of any suitable material, such as but not limited to stainless steel, Chrome-Cobalt, Nitinol, or another metal, or a polymer (nylon, PEEK, etc.) filament. Each tissue disrupting member 60a may have any suitable cross section (round, oval, rectangular or triangular), with a diameter of about 0.05-0.75 mm and a length of about 5-20 mm. Each wire or filament may be straight, curved or spiral along its length, with a smooth or roughened surface. In some embodiments, tissue disrupting members 60a may be configured to expand outward from a constrained configuration within shaft 14 to an expanded configuration when advanced out of distal end 17 of shaft 14. In some embodiments, such expansion may be achieved by constructing tissue disrupting members 60a at least partially from Nitinol, shape memory stainless steel, or any other suitable shape memory material. Once tissue disrupting members 60a have assumed their expanded configuration, tissue disrupting head 20a is rotated, in order to mechanically disrupt the mucosal lining of the fallopian tube. In alternative embodiments, tissue disrupting head 20a may include any suitable number of tissue disrupting members 60a, including fewer or greater than three.

FIG. 3B illustrates a tissue disrupting head 20b that includes three tissue disrupting members 60b shaped as ball-tipped tines. Tissue disrupting members 60b may be fabricated from any suitable material, such as but not limited to stainless steel, Chrome-Cobalt, Nitinol, another metal, or any suitable polymer (nylon, PEEK, etc.). Each tine of the tissue disrupting members 60b may have any suitable cross-sectional shape, such as round, oval, rectangular or triangular, any suitable diameter, such as about 0.25-1 mm, and any suitable length, such as about 5-10 mm. Each of the balls and the ends of the tines may be fabricated from the same material as the tines or from a different material, and may for example have a diameter of about 1-4 mm. Tissue disrupting members 60b may be angled at about 5-30 degrees relative to the longitudinal axis of tissue disrupting head 20b. The ball tips will contact the tissue when tissue disrupting head 20b is rotated, to mechanically disrupt the mucosal lining of a fallopian tube. In some embodiments, the tines of tissue disrupting members 60b may be configured to expand outward from a constrained configuration within shaft 14 to an expanded configuration when advanced out of distal end 17 of shaft 14. In some embodiments, such expansion may be achieved by constructing tissue disrupting members 60b at least partially from Nitinol, shape memory stainless steel, or any other suitable shape memory material. Once tissue disrupting members 60b have assumed their expanded configuration, tissue disrupting head 20b is rotated, in order to mechanically disrupt the mucosal lining of the fallopian tube.

FIG. 3C illustrates a tissue disrupting head 20c that includes a single, coil-shaped tissue disrupting member 60c. Tissue disrupting member 60c can be fabricated from a stainless steel, Chrome-Cobalt or Nitinol wire, or from a polymer (nylon, PEEK) filament. Tissue disrupting member 60c can have any suitable cross-sectional shape, such as round, oval, rectangular or triangular, may have a diameter of about 0.25-1 mm, and may have a length of about 5-20 mm. In one embodiment, tissue disrupting member 60c can be cone shaped, with a minimum diameter of about 0.5-1 mm, a maximum diameter of about 2-4 mm, and a length of about 5-20 mm. The pitch of the coil can be about 0.5-2 mm, with an angle of about 2-20 degrees. In order to prevent perforation of tissue surrounding the mucosal lining of the fallopian tubes, rotation of tissue disrupting head 20c is preferably opposite that of the coil winding. When rotated, tissue disrupting member 60c will contact the mucosal layer and disrupt this tissue. In some embodiments, tissue disrupting members 60c may be configured to expand outward from a constrained configuration within shaft 14 to an expanded configuration when advanced out of distal end 17 of shaft 14. In some embodiments, such expansion may be achieved by constructing tissue disrupting member 60c at least partially with Nitinol, shape memory stainless steel, or any other suitable shape memory material. Once tissue disrupting member 60c has assumed its expanded configuration, tissue disrupting head 20c is rotated, in order to mechanically disrupt the mucosal lining of the fallopian tube.

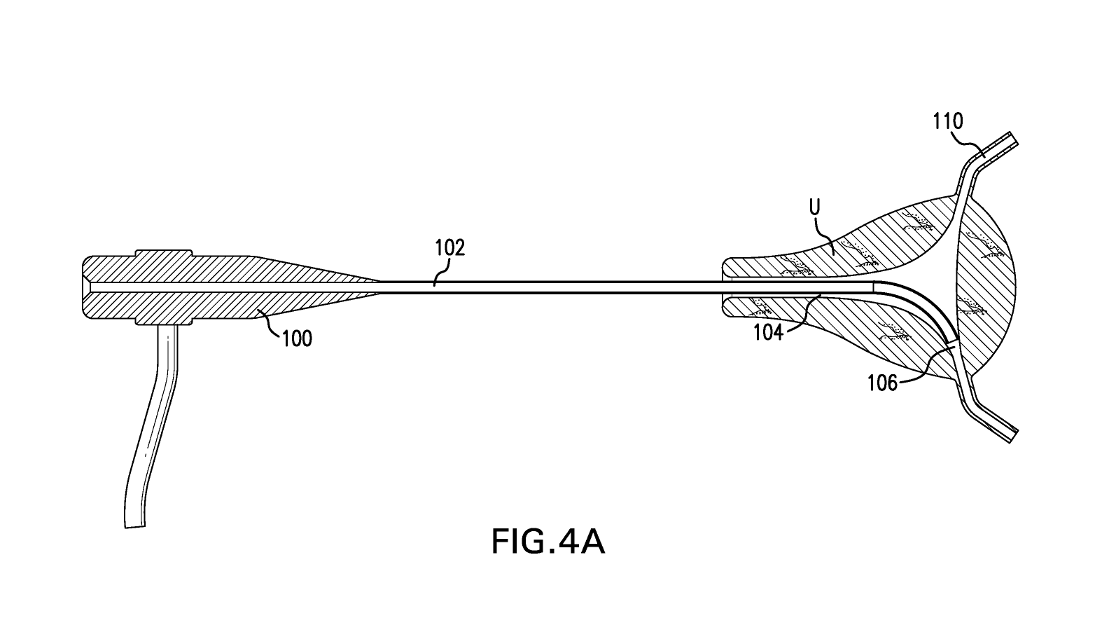

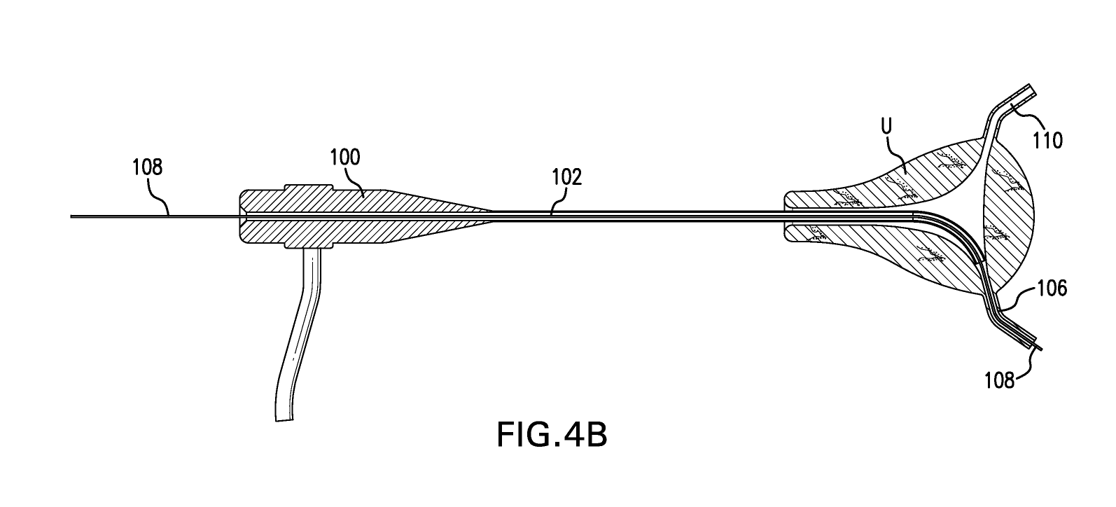

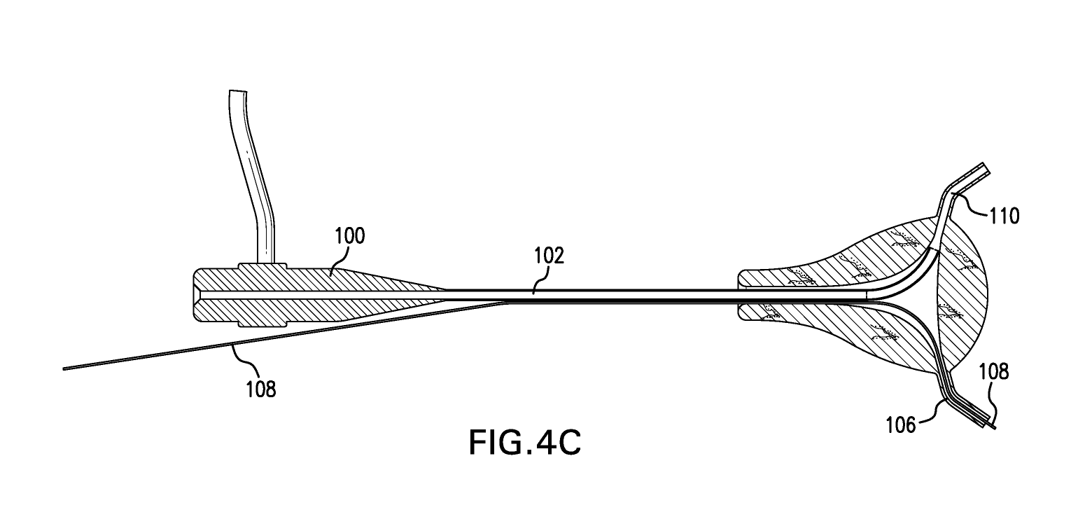

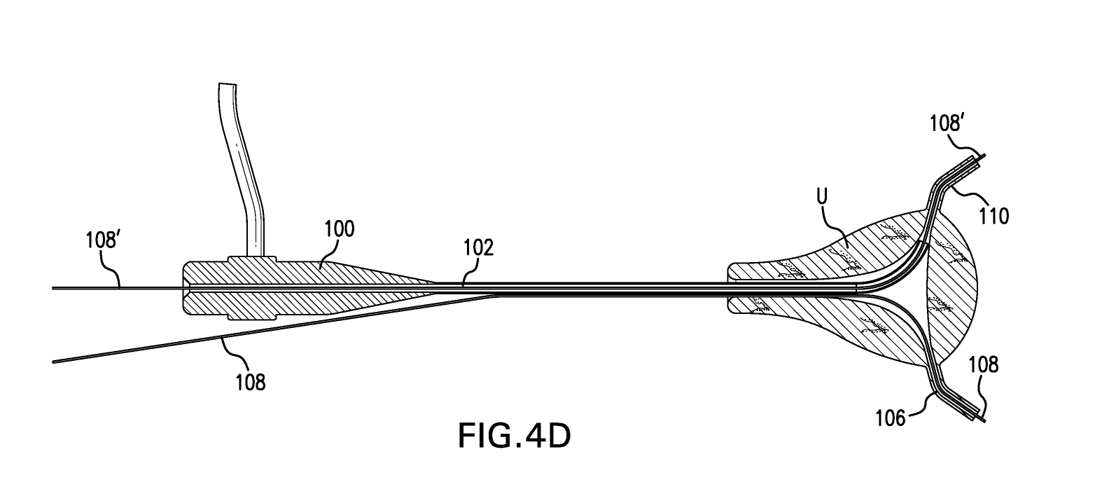

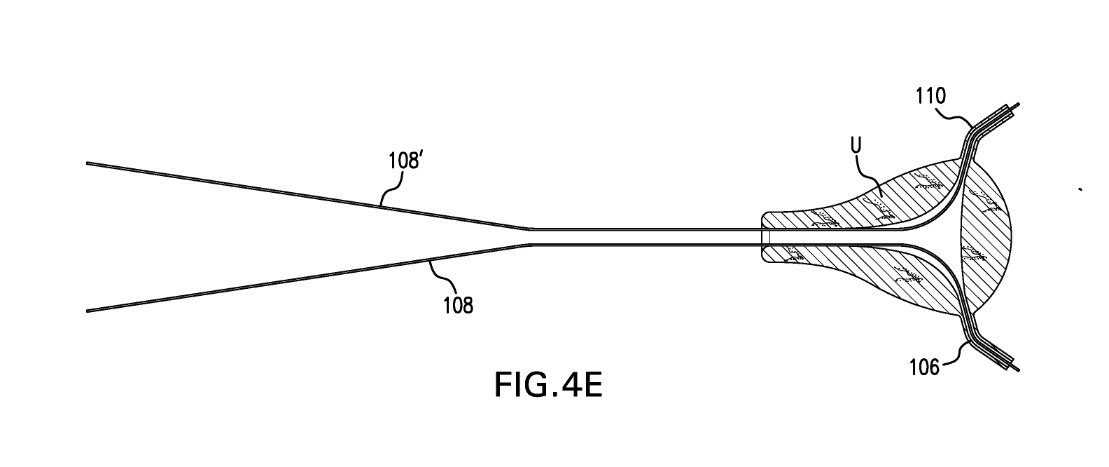

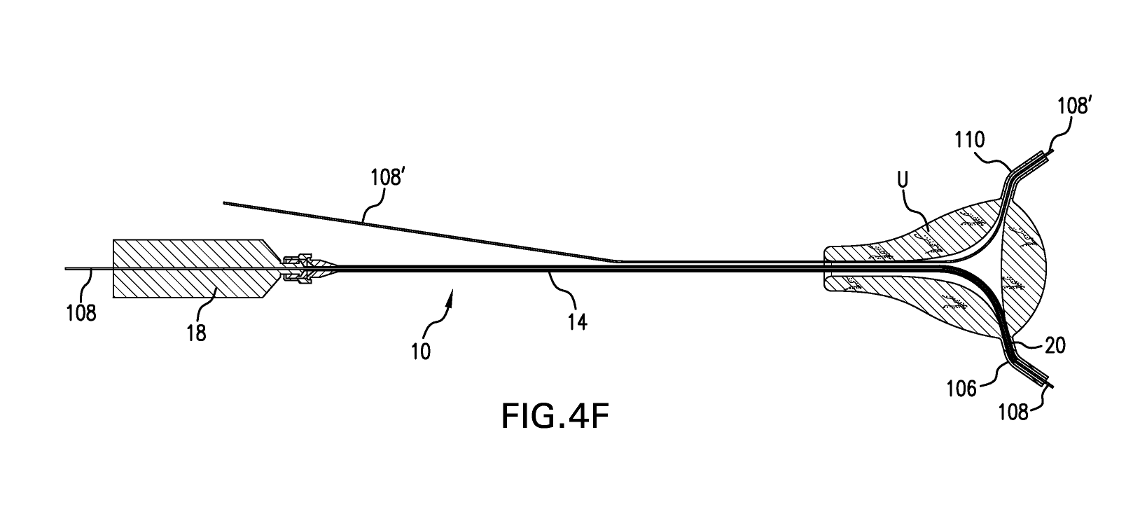

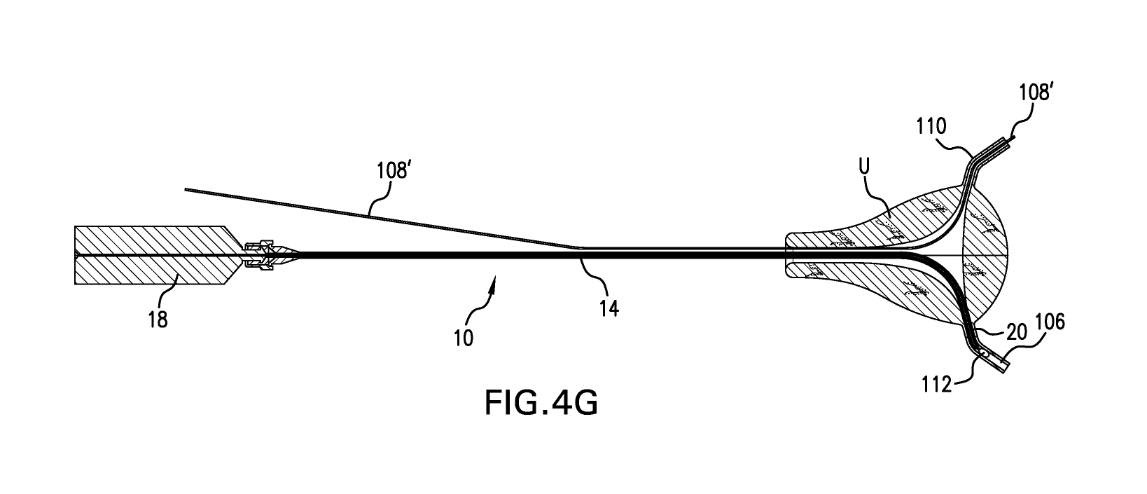

Referring now to FIGS. 4A-4G, a method for occluding the two fallopian tubes in a female patient, using fallopian tube occlusion system 10, is illustrated. The figures include a cross-sectional view of a uterus U, cervix 104, and two fallopian tubes 106, 110. Although this method includes addressing tissue in both fallopian tubes, and although in most embodiments both tubes will be addressed to promote contraception, in some embodiments system 10 may be used on only one fallopian tube.

As shown in the illustrated embodiment, a first part of the method may involve placing guidewires 108, 108′ in both fallopian tubes 106, 110, so that system 10 can be advanced over guidewires 108, 108′. In alternative embodiments, system 10 may be advanced into one or both fallopian tubes without the use of a guidewire. Therefore, the method of the present application should not be interpreted as being limited to use with guidewires. That said, and referring now to FIG. 4A, a first step in one embodiment may involve advancing a hysteroscope 100 having a working channel 102 through the vaginal canal and cervix 104, so that a distal end of the hysteroscope 100 is positioned in a first fallopian tube 106. Referring to FIG. 4B, a guidewire 108 may then be advanced through working channel 102 of hysteroscope 100 and into the first fallopian tube 106. Referring to FIGS. 4C-4E, this process may be repeated for the second fallopian tube 110 and a second guidewire 108′, and hysteroscope 100 may be removed, thus resulting in two guidewires 108, 108′ positioned within the two fallopian tubes 106, 110 (FIG. 4E).

Referring to FIG. 4F, system 10 may be advanced over guidewire 108 to position the distal end of shaft 14 in the intramural region of the first fallopian tube 106. In some embodiments, guidewire 108 may then be removed, before the fallopian tube procedure is performed, as illustrated in FIG. 4G. As shown in FIG. 4G, tissue disrupting head 20 may be advanced out of distal opening 17 of shaft 14 and operated as described above to disrupt the fallopian mucosal lining of the intramural region. For example, in various embodiments, handle 18, or a slidable portion of handle 18 coupled with tissue disrupting head 20, may be moved back and forth, proximally, distally, and/or rotated, to cause one or more tissue disrupting members on tissue disrupting head 20 to disrupt the mucosal lining of the fallopian tube 106. As part of the procedure, a sclerosant 112 is delivered from tissue disrupting head 20 and/or shaft 14 before, during and/or after tissue disruption with tissue disrupting head 20, as described above. The steps illustrated in FIGS. 4F and 4G, along with sclerosant delivery, may then be repeated for the second fallopian tube 110. System 10 is then removed, and the patient may be periodically (e.g., every several weeks) monitored for formation of tissue plugs in the fallopian tubes via hysterosalpingogram (HSG) or sonohysterography. In the latter procedure, sterile saline and air are injected into the uterus, and the physician looks for air bubbles passing through the fallopian tubes as an indication of patency.

Although various embodiments of systems and methods have been described in detail, these descriptions should not be interpreted as limiting the scope of the invention as it is defined in the claims. Any of a number of alternatives, modifications and variations of the described embodiments may be made, without departing from the scope of the invention. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is prior art to the present invention.

What is claimed is:

1. A system for treating a fallopian tube to promote contraception, the system comprising:
a catheter device, comprising:
a handle; and
a catheter shaft having a proximal end coupled with the handle and a distal end sized and configured to be advanced through a cervix and into the fallopian tube;
a tissue disrupting head coupled with the catheter and comprising at least one tissue disrupting member configured to mechanically disrupt a mucosal lining of the fallopian tube by contacting the tissue disrupting member with the mucosal lining and moving the tissue disrupting head; and
at least one fluid delivery port in at least one of the catheter shaft or the tissue disrupting head, for delivering a sclerosant to the mucosal lining of the fallopian tube at least one of before, during or after disruption of the mucosal lining by the tissue disrupting head.

2. The system of claim 1, wherein the tissue disrupting head is slidably disposed in a lumen of the catheter shaft, so that it slides in and out of a distal opening of the catheter shaft.

3. The system of claim 2, wherein the at least one tissue disrupting member is configured to disrupt the mucosal lining when the tissue disrupting head is moved back and forth relative to the fallopian tube.

4. The system of claim 2, wherein the handle comprises a sliding handle portion coupled with the tissue disrupting head, so that moving the sliding handle portion back and forth relative to the handle causes the tissue disrupting head to move in and out of the distal opening of the catheter shaft.

5. The system of claim 4, wherein the sliding handle portion is configured to rotate relative to the handle to cause the tissue disrupting head to rotate relative to the catheter shaft, and wherein the at least one tissue disrupting member is configured to disrupt the mucosal lining when the tissue disrupting head is rotated.

6. The system of claim 4, wherein the sliding handle portion is coupled with the tissue disrupting head via a tube slidably disposed within a lumen of the catheter shaft.

7. The system of claim 4, wherein the tissue disrupting head comprises a distal portion of a tube slidably disposed within a lumen of the catheter shaft and attached at a proximal end to the sliding handle portion.

8. The system of claim 2, further comprising a stop on an inner surface of the catheter shaft for stopping distal movement of the tissue disrupting head beyond a predetermined distance out of the distal opening of the catheter shaft.

9. The system of claim 1, further comprising a guidewire lumen extending through at least a portion of the catheter shaft.

10. The system of claim 9, further comprising a tube disposed within a lumen of the catheter shaft and connecting the tissue disrupting head to the handle, wherein the guidewire lumen comprises an inner lumen of the tube and extends from a proximal opening in the handle to a distal opening in the tissue disrupting head.

11. The system of claim 1, wherein the at least one fluid delivery port comprises a distal opening in the tissue disrupting head that is in fluid communication with a lumen of a tube extending proximally from the tissue disrupting head.

12. The system of claim 1, wherein the at least one fluid delivery port comprises multiple fluid delivery ports in a wall of the catheter shaft.

13. The system of claim 1, wherein the at least one fluid delivery port comprises multiple fluid delivery ports in the tissue disrupting head.

14. The system of claim 1, further comprising a quantity of the sclerosant for delivery through the fluid delivery port.

15. The system of claim 1, wherein the tissue disrupting head further comprises a tissue reservoir for collecting at least some of the mucosal lining that is disrupted.

16. The system of claim 1, further comprising at least one imaging marker on the catheter shaft for facilitating assessment of a location of the catheter shaft relative to the fallopian tube.

17. The system of claim 16, further comprising a tube slidably disposed within a lumen of the catheter shaft, wherein the tube further comprises at least one additional imaging marker.

18. The system of claim 1, wherein the catheter device further comprises a positioning member on an outer surface of the catheter shaft near its distal end, wherein the positioning member is configured to prevent advancement of the distal end of the catheter shaft past a desired location in the fallopian tube.

19. The system of claim 1, wherein the at least one tissue disrupting member is selected from the group consisting of wires, coils, tines with attached balls, and blades.

20. A method of occluding a fallopian tube, the method comprising:
advancing a distal end of a catheter into the fallopian tube;
positioning a tissue disrupting head coupled with the catheter in an intramural region of the fallopian tube;
moving the tissue disrupting head within the intramural region to mechanically disrupt a mucosal lining of the fallopian tube; and
delivering a sclerosant to the mucosal lining of the fallopian tube.

21. The method of claim 20, wherein advancing the distal end of the catheter comprises advancing it through the cervix to access the fallopian tube.

22. The method of claim 20, wherein positioning the tissue disrupting head comprises advancing the tissue disrupting head out of the distal end of the catheter.

23. The method of claim 22, wherein advancing the tissue disrupting head out of the distal end of the catheter allows one or more tissue disrupting members on the tissue disrupting head to expand from a constrained configuration to an expanded configuration for disrupting the tissue.

24. The method of claim 20, wherein moving the tissue disrupting head comprises at least one of moving the tissue disrupting head proximally relative to the fallopian tube, moving the tissue disrupting head distally relative to the fallopian tube, moving the tissue disrupting head back and forth, or rotating the tissue disrupting head.

25. The method of claim 20, wherein delivering the sclerosant is performed at least one of before, during or after moving the tissue disrupting head to disrupt the mucosal lining.
